(19) United States
(12) Patent Application Publication
(10) Pub. No.: US 2017/0112412 A1
(43) Pub. Date: Apr. 27, 2017

(54) DETECTING AIR PRESSURE AND RESPIRATORY CONDITIONS USING A DEVICE TOUCH SENSITIVE SURFACE (TSS) SUBSYSTEM AND OTHER BUILT IN SENSORS SUBSYSTEMS

(71) Applicant: Raymond J. Garbos, Mont Vernon, NH (US)

(72) Inventor: Raymond J. Garbos, Mont Vernon, NH (US)

(21) Appl. No.: 15/296,184

(22) Filed: Oct. 18, 2016

Related U.S. Application Data
(60) Provisional application No. 62/246,295, filed on Oct. 26, 2015.

(51) Int. Cl. A61B 5/087; G06T 11/20; G06F 3/0346; A61B 7/00; G06F 3/16; G06F 3/0354; G01L 19/00; A61B 5/00; G06F 3/041; G06F 3/01

(52) U.S. Cl. CPC A61B 5/087; G06F 3/0416; G06T 11/206; G06F 3/0346; G06F 3/011; G06F 3/16; G06F 3/03545; G01L 19/0092; A61B 5/6898; A61B 5/7282; A61B 5/743; A61B 5/7475; A61B 7/003; G06F 2203/0381; G06T 15/00

(57) ABSTRACT

Techniques for using the sensor subsystems built into typical smart devices such as a smartphone to detect, measure, collect and characterize air pressure waveforms and make recommendations. The approach is particularly adaptable to detecting respiratory conditions but may also be used in other applications.

### CROSS-REFERENCE TO RELATED APPLICATIONS

The present application claims the benefit of U.S. Provisional Patent Application Ser. No. 62/246,295 filed on Oct. 26, 2015, by Raymond Garbos for a DETECTING RESPIRATORY CONDITIONS USING PORTABLE DEVICE and is hereby incorporated by reference.

### BACKGROUND

Technical Field

This patent application relates to sensing air pressure and/or vibration, using a touch sensitive surface subsystem, accelerometer(s) subsystem and/or microphone subsystem with a processing subsystem and/or other subsystems built into a smartphone, tablet or similar smart device.

Summary of Related Art

Touch Sensitive Surfaces (TSS) have revolutionized the way a user interfaces to smart devices. Smart devices are devices that have some internal processing/computer ability to process information. Smart devices include mobile devices (e.g. smartphones, tablets, portable computers, etc.) and fixed devices (e.g. workstations, ATMs, kiosks, control panels, etc.). The most common TSS built into many of these smart devices is a touch screen or a touch display. The TSS eliminates the need for a keyboard, cursor and/or computer mouse. A user, using a finger or mechanical stylus, contacts the touch sensitive surface and thus provides a pressure/force on a “spot” on the touch screen sensing array. The TSS subsystem detects the pressure/force, calculates the location and provides that information to the internal processing subsystem of the smart device which uses its software to make decisions. Because smart devices have electrical input/output connectors, inventors have designed external measurement devices or special hardware that can be used to provide additional information. However, these are extra devices/hardware at an additional cost and are not used by the majority of the population owning or using smart devices. The invention applied for in this patent application does not require the user to physically touch the display or obtain additional hardware to measure the air pressure.

### SUMMARY OF THE INVENTION

In one example implementation, a user coughs, wheezes, or otherwise directs their breathing at a smart device's touch sensitive surface. The pressure of the cough activates the appropriate existing built in sensor subsystems—such as the touch screen, accelerometer(s), motion detector(s), and/or microphone. The detected signals can be processed and/or combined in various ways to analyze and/or diagnose certain respiratory or other conditions and determine if a condition is about the same, getting better, getting worse or critical.

Using only the mobile/portable device and its built-in subsystems (touch screen, motion detectors, microphone, Radio Frequency (RF) Communication—(WiFi, internet (e.g. 4G) and phone) and software running on the device and/or connected server can measure, store, process and develop a multi-sensor three-dimensional (3D) vs. time image that software can process and classified the patient's condition. This invention enables a smart device like a smartphone or tablet to become a low-cost mobile/portable medical tool to aid the user and their physician in adhering to their medical plans by measuring, identifying, evaluating and then provides the user with a pre-established action plan(s) associated with certain medical conditions such as respiratory conditions.

In another example implementation, an (adjustable) air pressure stylus is developed and incorporated as a Human-to-Device or Machine/Robot-to-Device input device that utilizes the air pressure sensing of the TSS associated with this invention. With an adjustable air pressure stylus, the user can adjust the orifice area (breath) to affect a larger footprint and the pressure resulting in more resolution (depth) at the “spot”. This will provide game and other mobile software App developers more flexibility then current finger or fixed stylus contact.

In different example implementations:

1. The Touch Sensitive Surface (TSS) sensor subsystem can be used to measure a pressure wave from the user's breathing, wheezing and/or coughing into the device, and can be further used to develop a 3D image of such an air pressure wave.
2. Built in motion detector sensors (accelerometers) subsystems can be used to measure the force of a pressure wave, for example human/animal breathing, wheezing and coughing, or other airflows, and can also be used to develop a two-dimensional (2D) image of the wave.
3. Built in motion detector sensors (accelerometers) subsystems can be used to measure vibrations when the smartphone or similar device is in actual contact with a surface (for example when in contact with the human body). The same may be used to measure other vital signs such as pulse rate and breathing rate and can also be used to develop a 2D image of these waveforms.
4. Built in microphone(s) or other sensor subsystems can be used to simultaneously detect the audio or acoustic signals associated with 1, 2 or 3 above.
5. The images and waveforms developed with 1, 2, 3 and/or 4 above can be used individually or in various combinations to provide a multi-dimensional, multi-sensor description of the pressure wave.
6. A battery or powered adjustable air pressure stylus can be used as a Human-to-Device or Machine/Robot-to-Device input device instead of coughs, etc. It would create images and waveforms like those developed with 1 and 2. This will provide game and other mobile software App developers more data and flexibility then current finger or fixed stylus contact. This air pressure stylus may also be of use for people with disabilities so they can interface with these commercially available devices or other touch sensitive devices e.g. ATMs.
7. A specific medical example application of this sensing capability can be to have a patient breathe or cough so the device can measure conditions such as respiratory ailments (ex. Asthma and COPD—Chronic Obstructive Pulmonary Disease) and the like.

### DESCRIPTION OF THE DRAWINGS

The following drawings are included to provide a better understanding of the invention, using a smartphone as an example. These drawings are part of the application and along with the descriptions serve to describe the invention.

FIG. 1—Typical Smartphone with Touch Sensitive Surface (TSS)—is a picture of a typical commercially available smartphone circa 2015.

FIG. 2—Typical Smartphone Block Diagram—is a diagram showing the various subsystems available in a typical smartphone.

FIG. 3—Touch Screen Display Heat Map—is a drawing of the touch array response as it is activated by applying pressure by a finger touching the array.

FIG. 4—User Coughing onto a Smartphone TSS—is a drawing of one instance of how this invention gets activated.

FIG. 5—Cough Analysis Processing Flow Diagram—is a flow chart of how a smartphone processes the invention to benefit the user.

FIG. 6—Acoustic Waveform of a Breath—is the result of a test to demonstrated that the acoustic waveform of a breath is detectable and measurable.

FIG. 7—TSS Cough Response vs. Time—X axis—is a representation of the touch screen response over the duration of a cough for the X axis.

FIG. 8—TSS Cough Response vs. Time—Y axis—is a representation of the touch screen response over the duration of a cough for the Y axis.

FIG. 9—TSS Cough Response Waveform Slice at Time T—is a 2 dimension (X, Y) representation of the touch screen response at sample time=T during the cough.

FIG. 10—Smartphone Z-Axis Accelerometer Waveform of Same Cough—is a representation of the waveform of the z-axis accelerometer response for the same cough in FIGS. 7, 8 and 9.

FIG. 11—Smartphone Microphone Waveform of Same Cough—is a representation of the waveform of the microphone response for the same cough in FIGS. 7, 8 and 9.

FIG. 12—Accelerometer Waveform of a Cough with Smartphone on Chest—is a representation of the result of a test to demonstrate that the smartphone accelerometers can detect vibrations associated with a hearth beat and breathing rate.

FIG. 13—Air Pressure Stylus Activating the TSS—is a concept drawing of an adjustable air pressure stylus invention that takes advantage of the air pressure sensitivity of the touch screen to provide more flexibility to the user than a finger or standard touch screen stylus.

### DETAILED DESCRIPTION OF AN EMBODIMENT

In example implementations, the built-in features/subsystems of portable/mobile devices such as smartphones, tablet computers, mini-pads, smartwatches, etc. or other devices having any touch sensitive surface are used for detecting air pressure waves, vibrations, etc.

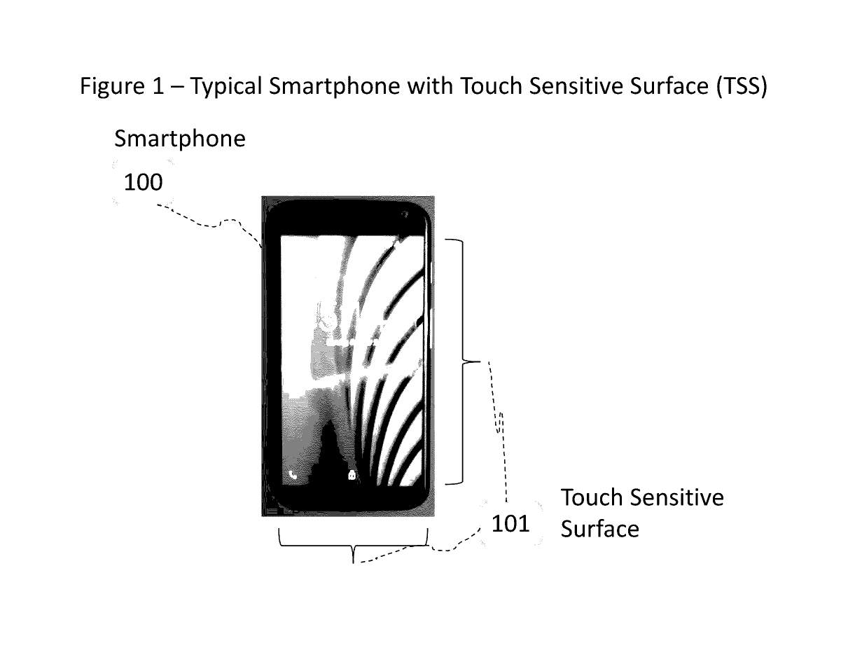

FIG. 1 illustrates a typical smartphone device 100 such as a Motorola® Moto X® (Motorola and Moto X are trademarks of Motorola Trademark Holdings, LLC of Chicago, Ill.) having a bar/slate form factor where a display is the dominant feature of the front face. The key device's built in features/subsystems include:

for input—touch screen 101, Micro Electrical Mechanical Systems (MEMS) acceleration 3 axis motion sensors or gyros 202 (used in a typical smartphone for portrait/landscape screen orientation and/or as controller inputs for video gaming), microphone 203, and other input functions 204 such as RF/InfraRed (IR) receivers, cameras; and

for output—functions 205—such as display, audio speaker, vibrations, and RF/IR transmitters.

The approach is particularly useful for patient-centered measurements to help with ehealth/mhealth diagnostics, maintenance and care including tele-medicine. With the addition of the adjustable air pressure stylus, application (app) developers will be able to incorporate more accuracy and more resolution per activation thus providing more information.

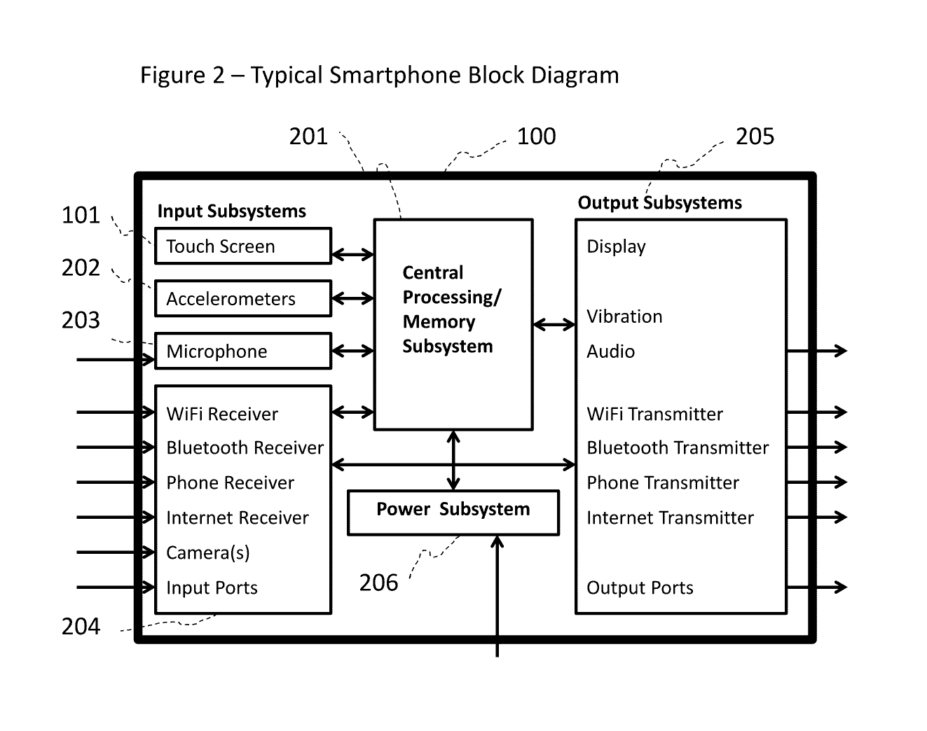

FIG. 2 is a detailed block diagram of the typical smartphone device 100 subsystems, showing the basic features/subsystems already built into such smart devices. A subsystem includes all the hardware and software/firmware necessary to implement a feature. The key sensor subsystems for this invention are the touch sensitive surface 101, 3-axis accelerometers 202 and microphone 203. These smart devices can internally process data via the processing subsystem 201 but are also able to wirelessly communicate in and out of the smart device and/or establish data connections 204, 205 to send the detected signals to user, emergency personnel and/or other caregivers. The smart devices can also interface with the Internet of Things (IoT) providing access to other apps, information, storage and processing resources ad facilitating eHealth and tele-medicine. The smartphone 100 provides information to the user using output subsystem 205 functions like the display, audio speakers and vibrations. The smartphone 100 also has an internal power/battery subsystem 206 that provides power to the various internal subsystems as well as external devices that may be connected to the smartphone by its electrical/mechanical Input/Output (I/O) ports 204, 205. The Input/Output (I/O) ports 204, 205 can also be used to transmit/receive data/signals over a cable.

This invention can result in using a smart device as a personal medical tool to improve the quality of life, reduce medical visits and costs, and improve productivity of users/patients, as well as user/patient adherence to a medical plan, facilitation of self-management and patient-nurse-physician-caregiver-pharmacist communication.

In one example implementation, the smartphone 100 device incorporates a mutual capacitance touch screen 101 that uses a projected-capacitance (pro-cap) electrode grid. The intersections of the electrodes in the grid form a mesh of touch sensitive points located between two conductor sheets under the touch surface mesh. The mesh typically has a size of typically 4 to 8 millimeters (mm) or more and can be sampled at rates over 200 samples per second. It also contains a thin bearing plate. They are designed to have a user touch and apply pressure on a “small” area of the screen with their finger or a mechanical stylus.

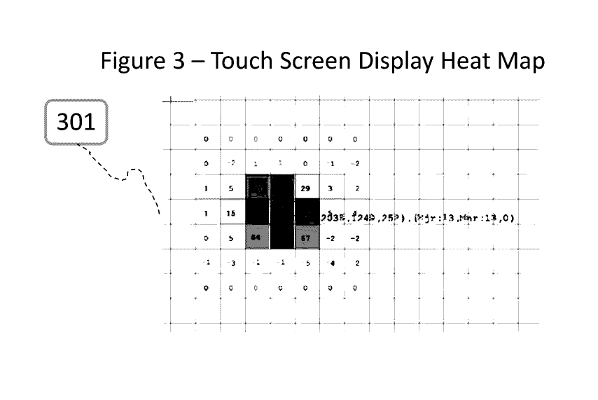

The touch sensing grid 101 may provide a set of signal levels detected by a number of unit cells. In one embodiment, each unit cell may be associated with an intersection between unique pairs of horizontal and vertical sensor elements, with the signal levels representing the difference between a value measured at the corresponding grid intersection and a baseline value for the array. A resulting “heat map” 301 (FIG. 3) represents a location/pressure footprint of a finger or stylus that touched the screen surface.

Other types of touch sensitive screens 101 may also be used, such as those that additionally can detect microscopic differences in distance between the cover glass and the backlight as pressure is applied to the screen.

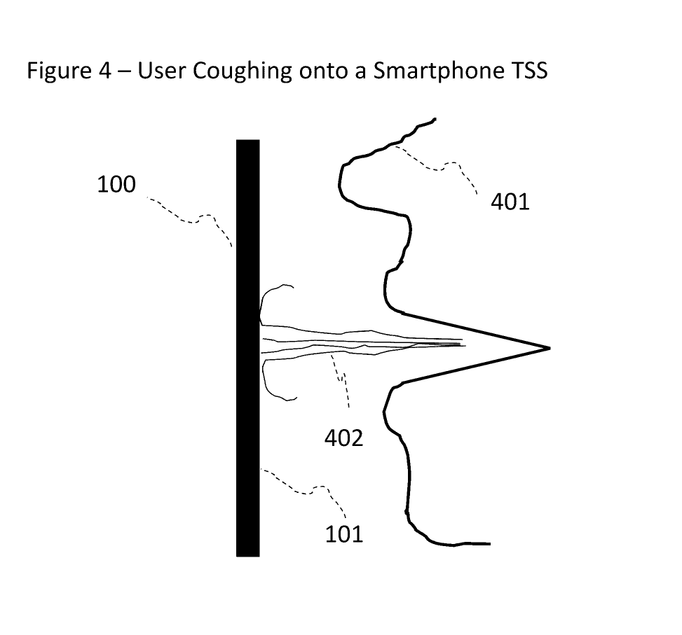

In one example of detecting an air pressure wave using a touch sensitive surface 101 is shown in FIG. 4. A user 401 coughs and creates an air pressure 402 on the smartphone 100 touch sensitive surface 101. An individual is directed to cough and/or breathe on the device screen (with the touch sensitive surface 101 in front of their mouth, but without actually coming into physical contact with the screen).

The touch screen 101 subsystem will then respond and provide measurements from the touch sensitive mesh array. This response is then processed by the processing subsystem 201 to create a sequence of 2D “images”, for example, with one 2D image taken each 50 microseconds for duration of the cough or breathing. These 2D images can then be combined to create a 3D image of the volume vs. time representative of the cough/breath pressure projected onto the touch screen. In one test for proof of concept of the invention, signals were sufficiently detected by breathing onto a typical Samsung® touch screen display 101 (Samsung is a trademark of Samsung C&T Corporation of Seoul, Korea).

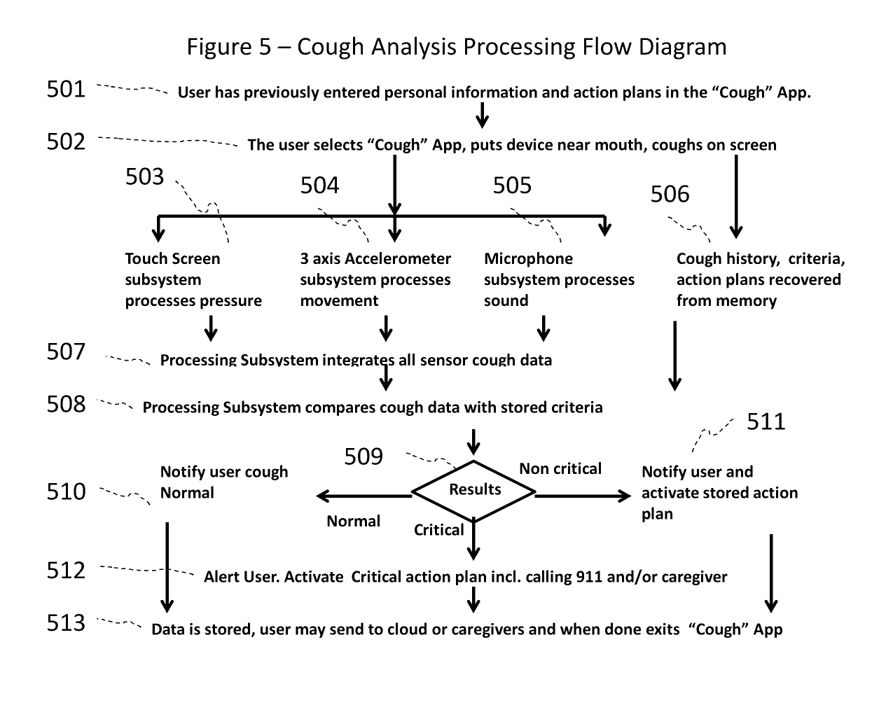

FIG. 5 is a chart of the cough analysis processing flow of the smart device. This processing would typically be implemented as a software/firmware application, e.g. a downloadable program called “Cough” App could be implemented in hardware/firmware in some situations.

When the user initially downloads and activates the “Cough” App, the user enters personal information, and, working with the user medical team, enters existing medical data and various action plans and decision criteria 501. When the user is ready to use the “Cough” App 502, the user selects the App on the smartphone 100, follows the instructions and coughs on the Touch Screen 101 like shown in FIG. 4. All the smartphone subsystems shown in FIG. 2 are involved in some way. The Touch Screen Subsystem 202 responds to the pressure wave by sampling the array many times a second creating a series of 2D images 503. Simultaneously the 3 axis Accelerometers detects and measures the force of the pressure wave which causes a slight physical movement 504. Simultaneously, the Microphone detects and measures the acoustic waveform of the pressure wave 505. When the “Cough” App is activated various information like cough history, decision criteria, action plans 506 may be available in the local storage area or may be downloaded via the input 204 and output 205 communication subsystems and be available to the processing subsystem 201.

The processing subsystem 201 integrates/fuses the sensor data 507 from one or more of the sensors to create a 3D multi-sensor representation of the cough. This may involve calculating key decision criteria including volume, velocity, duration, abnormalities etc. The processing subsystem 201 compares the real time cough parameters with those previously stored by the user 508. The processing subsystem 201, using the previously stored criteria, processes all data leading to a result 509. There may be many possible results: FIG. 5 shows three possible results. One result 510, where the results fall within a Normal criteria. The user is notified via one or more of the output subsystems features and provided with recommendation via the Normal action plan. A second result 511, the user is notified that the cough is bad or the condition has degraded and is provided with recommendation(s) via that action plan. A third result 512, the user is notified that the cough condition is very serious and requires immediate action and is provided with recommendation(s) via that action plan, including automatic calling to a care giver or 911. For all results, all the initial sensor data and all calculations are saved 513 (locally and/or to a remote storage area) to be available for future analysis and digitally sent via user approval to physician. At the end the user exits the “Cough” App allowing the device to be used for other functions.

Prior to or immediately after the cough, the built-in MEMS 3 axis accelerometers/motion detectors 202 may be used to measure the user's heart and breathing rate. These three (3) axis accelerometers 202 used in a typical smartphone 100 are able to measure acceleration with a minimum full-scale range of ±3 g. These accelerometers measure the static acceleration of gravity in tilt-sensing applications, as well as dynamic acceleration resulting from motion, shock, or vibration. Bandwidths vary slightly by the portable/mobile device manufacturer, but typically have a range of 0.5 Hz to 1600 Hz for X and Y axes, and a range of 0.5 Hz to 550 Hz for the Z axis.

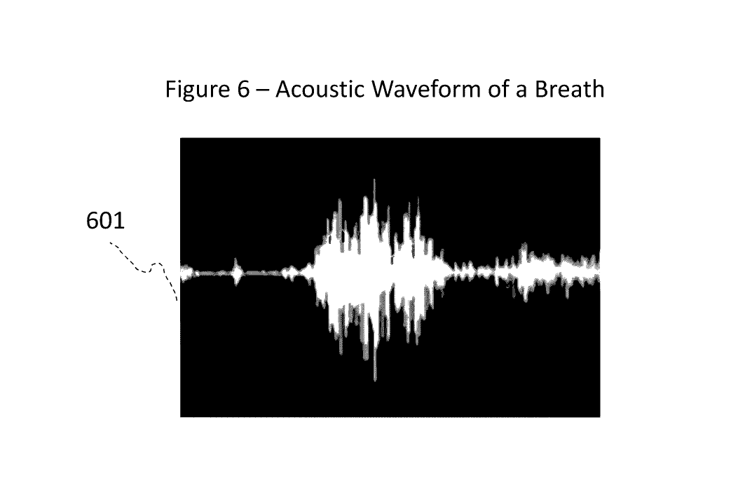

The built-microphone 203 may be used to record the ambient sound of the cough to create an acoustic waveform over the same timeframe as the detected pressure wave. A typical microphone can detect frequencies from 0 to 40 kHz (kiloHertz) which is sufficient to capture most breathing waveforms of interest. FIG. 6 is an acoustic waveform 601 of a normal human breath to demonstrate that a microphone can detect a coughing sound.

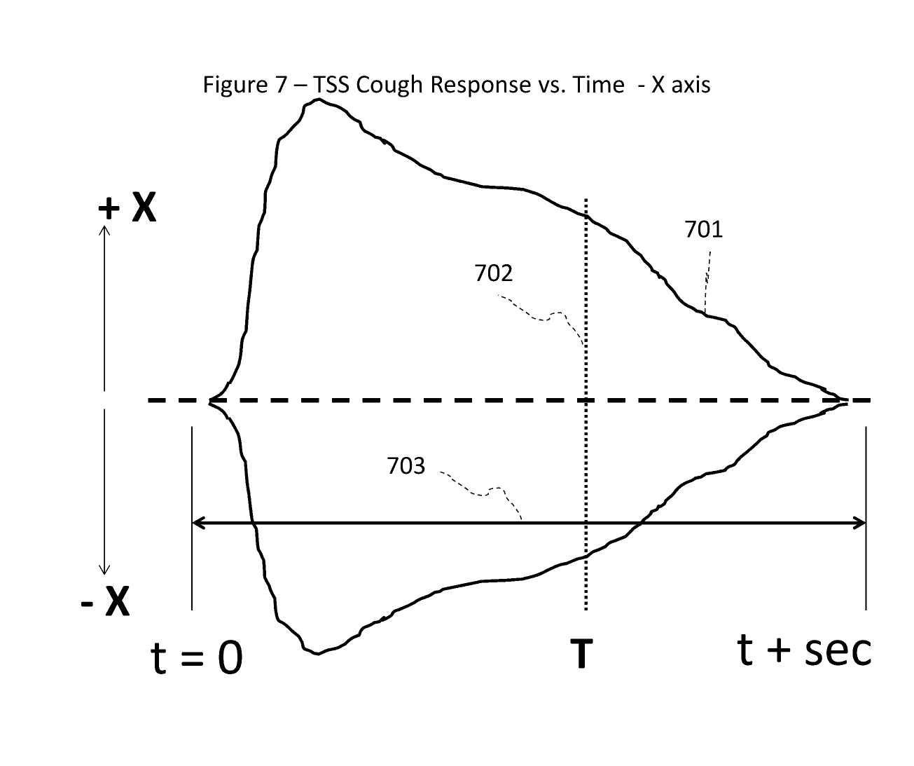

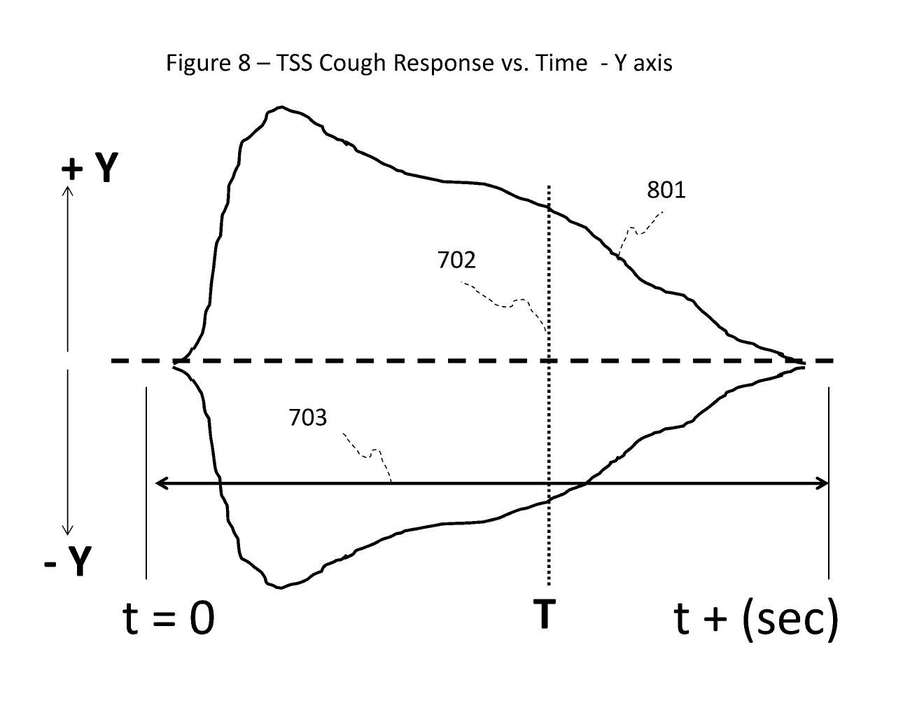

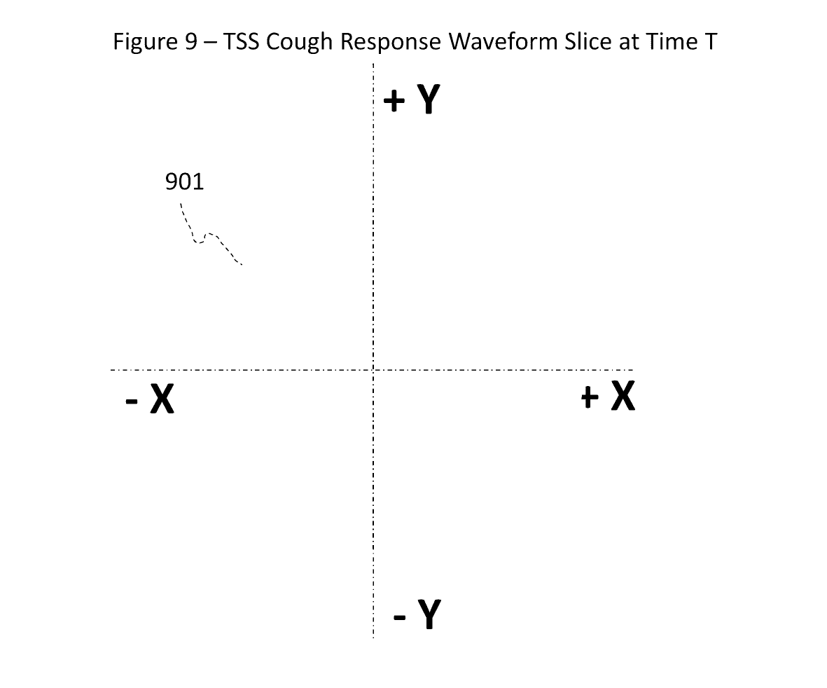

FIGS. 7, 8, 9, 10, 11 are depictions of how the three sensors (touch screen 101, accelerometers 202 and microphone 203) may respond to a cough 402 from a user 401. FIGS. 7, 8 and 9 are measurements from the touch screen pressure grid 101. FIG. 7 shows the cough waveform 701 which is the X axis centered on touch screen 101 over time 703, FIG. 8 shows the cough waveform 801 which is the Y axis centered on display over time 703 and FIG. 9 is a snapshot of one of the 2D sample 901 at Time “T” 702 centered on the touch screen array 101. From this information, one can develop not only a calculation of the volume of the cough but the shape during the cough and the force over time. All of these 2D and 3D waveforms will be useful in determining the condition of the user in real time by the processing subsystem 201 as well as providing data for remote storage and processing via the smartphone output subsystem 205.

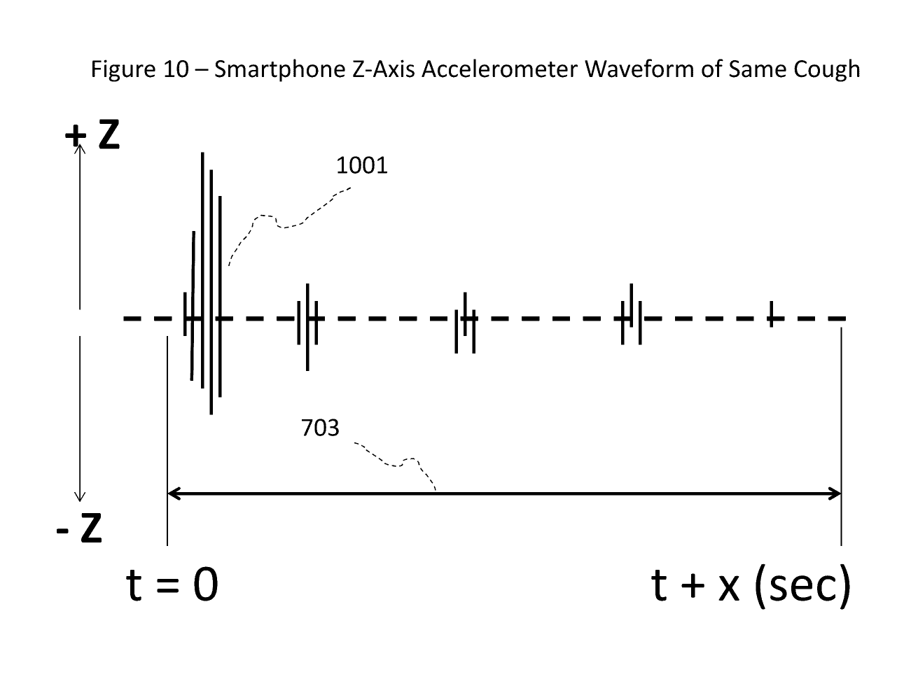

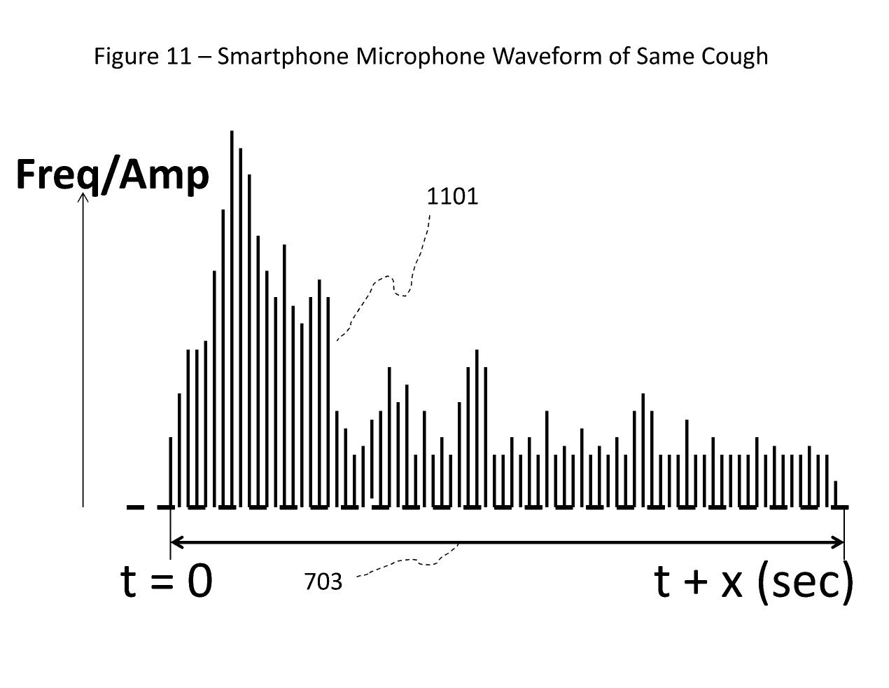

FIG. 10 depicts the Z axis accelerometer 202 waveform 1001 for the same cough 402. FIG. 11 depicts the microphone 203 acoustic waveform 1101 for the same cough 402.

By integrating the data from the three sensors (touch screen 101, accelerometers 202 and microphone 203), an information rich multi-sensor image of a cough can be developed and analyzed to help the user and physician manage the patient's condition.

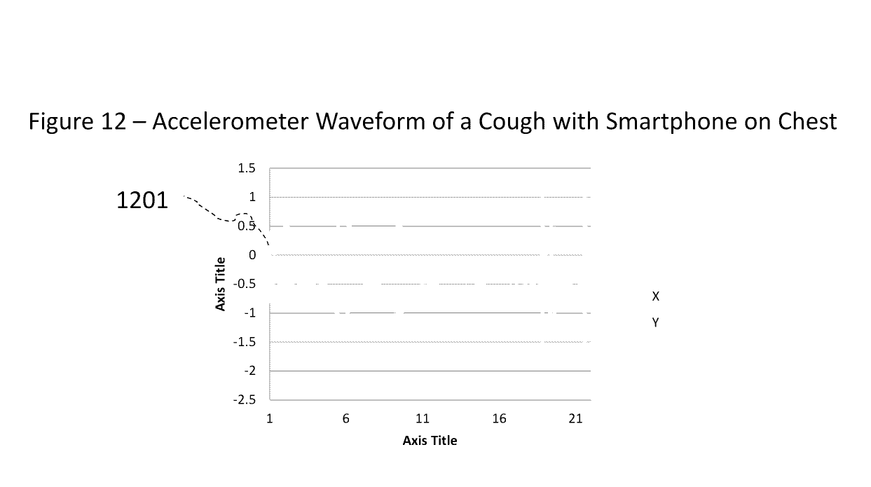

An additional input representing the person's heart/pulse rate can be measured by placing the portable/mobile device 101 on their body (e.g. chest or wrist). Taken immediately before or after the cough is measured, this additional information can indicate whether the user's physically active caused an Asthma or COPD incident, thus providing additional real time diagnostic data. In one test shown in FIG. 12 the accelerometers 202 on a Samsung smartphone where shown to provide a detectable cough waveform 1201. Other tests have shown that breathing rate may also be detectable, if the phone is touched to the body.

These three smartphone sensor subsystems—touch screen 101, accelerometers 202 and microphone 203 may be integrated together using signal processing and/or pattern recognition algorithms. The result may provide a very unique 3D multi-spectrum image over duration of the cough/breath. These images can then be stored and processed in near real time to determine if the patient's conditions are normal, getting better or worse or critical, and can be used to create warnings or recommendations and enable real time tele-medicine. The stored waveform can then be provided with patient approval to a physician/caregiver for post diagnostics.

It will be understood that the “smartphone” 101 mentioned above could also be a tablet, mini-pad, Apple iPad®, Microsoft Surface®, or another similar device having built-in touch screen or surface, accelerometer(s) and/or microphones.

The following table shows various respiratory measurements and/or actions related to those measurements that Asthma or COPD patients and caregivers may require. It also shows the various smart devices' built in capabilities that are used to make those measurements or actions enabled by the ability of the device to measure and analyze the air pressure from breathing/coughs.

| Respiratory Related | Smartphone | |
|---|---|---|
| Measurement | Feature | Comment |
| Breathing sound and rate (including | Microphone, | Frequency, amplitude, time plots and recognition |
| during sleep) | touch display, | algorithms, pressure on display, movement of phone |
| Respiratory rate (# breaths/minute) | movement sensors | accelerometers. |
| Airway sounds (e.g. cough, wheeze, | | |
| stridor, rhonchi) | | |
| Congestion: lower vs. upper respiratory | Microphone | Frequency, amplitude, time plots and recognition |
| congestion | | algorithm |
| Crackles, Coarse rhonchi, Polyphonic | | |
| wheeze (positional) | | |
| Pulmonary Function Test | Microphone, | Frequency, amplitude, time plots and recognition |
| Lung Function Test | touch display, | algorithm, movement of phone |
| Spirometer Test (FEV1, FVC, | movement sensors | |
| FEV1/FVC) | | |
| Peak Flow | | |
| Heart Rate, Breathing Rate | Touch display, | Pressure on display, movement of phone touched to |
| Heart (or pulse) rate +/− rhythm | movement | body, radar reflections from RF transmitters/receivers |
| (regular vs. irregular) | sensors, RF | |
| | communication | |
| | subsystem | |
| Activity/Movement (household, | Video Camera, | Stored for Physician review, w/w interface to |
| exertion, sport, fall. etc.) | movement | wearable devices (e.g. sport bands, health tags) also |
| Physical activity level/movement | sensors, IOT | measures heart and breathing rates. |
| (awake - rest/exercise, sleep) | | |
| Local Air Environment (wind, | GPS location and | GPS locator (integrated with local weather data). |
| temperature, smog, smoke, humidity, | existing IOT | Provide warnings, plus stored for post cluster analysis |
| pollens, sand, dust, etc.) | information | by physician and/or expert system. |
| Air quality and ‘climate’ (via GPS, | | |
| weather, EPA) | | |
| Incident Trigger (precipitating event | Video Camera, | GPS locator (with maps data). Stored for post analysis |
| stress, anxiety, environment, etc.) | GPS location and | to determine where patient was prior to attack that |
| Asthma trigger, allergen or | existing IOT | may have caused it. |
| exacerbating factor | information | |
| Prescription/OTC Meds adherence | Alarm (audio, | Event calendar - initial notice with follow up |
| monitoring, side effects, proper medical | vibration, screen | prompts, text to caregiver and/or physician if |
| device use | warning/flashing), | sufficient overdue. Track prescription refills including |
| Medication list | Input via touch | needed renewals and respiratory related visits. Provide |
| | screen and voice. | common side effects. How to Videos for medical |
| | | devices |
| Individual ‘classifications’ | Storage, internet | Physician should determine appropriate patient |
| Personal risk profile and social | access | specific categories: age, gender, ethnicity, general |
| characteristics affecting asthma | | health, pregnancy, family history, local concerns, |
| outcomes and barriers to | | hospital/ER visits etc. |
| change/usability of features | | |
| Caregiver, Emergency real time contact | email, phone | 911 like capability that can provide location and |
| Emergency contact and consent to | | important medical data pre-approved by patient |
| share/access patient-specific data (+911) | | |

Although the approaches described herein have specific applicability to detecting respiratory conditions in a human, they might also be used similarly in animals. It may also find use in other applications where the ability to easily characterize a pressure wave is helpful. These uses may include, but are not limited to, detecting and measuring other types of air pressure waves, such airflows or air leaks occurring in equipment or other industrial settings, detection of wind speed and/or wind direction, and other applications.

If an adjustable air pressure stylus is also incorporated as a Human-to-Device or Machine (Robot)-to-Device interface, then the number of cells impacted on the grid can be greatly reduced improving accuracy and resolution. As important, the sensitivity or pressure and response can be adjustable. Currently with a finger or conventional manual stylus one is able to touch the screen but only determines two states when the touch screen is activated—soft or hard. With an adjustable air pressure stylus many other states can be obtained offering game and other app developers more flexibility. It may also be used by people with disabilities to more easily interface with touch screen devices including not just smartphones, tablets, etc., but also ATMs, ordering terminals/kiosks currently showing up at fast food centers, malls etc.

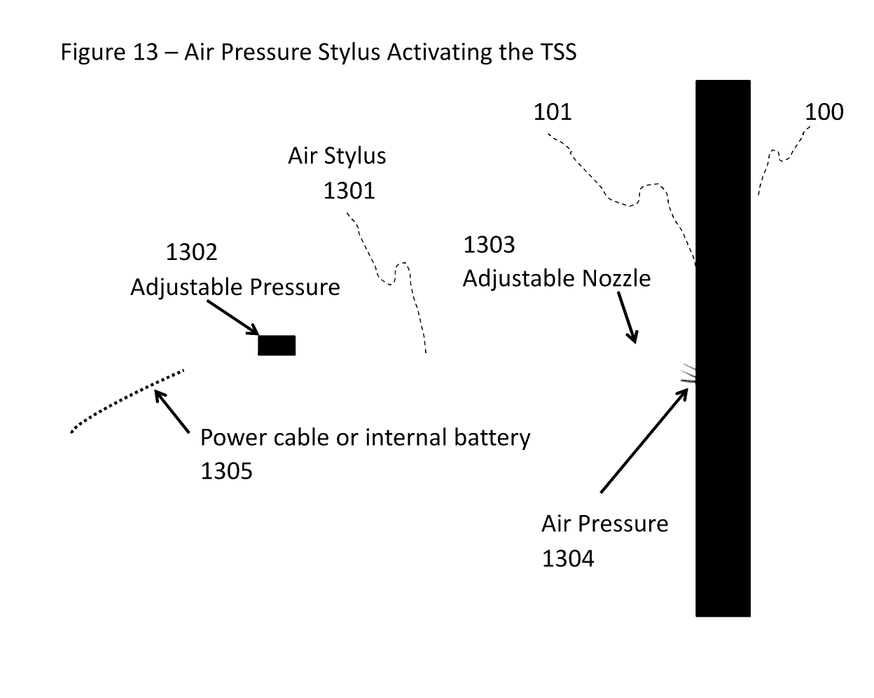

FIG. 13 depicts an example air pressure stylus 1301 where the user is able to activate the touch screen 101 via a pressure wave 1304 and with optional features to adjust the pressure 1302 and the orifice area 1303. Adjustments may be local or remotely controlled. The air pressure stylus may be battery operated/charged or powered via cable 1305.

What is claimed is:

1. A method comprising:
detecting one or more outputs from a touch screen of a device generated by an airflow impinging on the touch screen; and
processing the output(s) to characterize the airflow.

2. The method of claim 1 additionally comprising:
combining the one or more touch screen output(s) detected over time to provide a three-dimensional representation of an air pressure wave.

3. The method of claim 1 wherein the outputs of the touch screen comprise an array of data points representing sensed pressure at each of a plurality of positions within sensor grid of the touch screen.

4. A method comprising:
detecting one or more outputs from motion sensor(s) of a device generated by a user breathing on or near the device; and
processing the output(s) to characterize a respiratory condition of the user.

5. A method comprising:
detecting one or more outputs from a touch screen of a device generated by a user breathing on the touch screen;
detecting one or more outputs from motion sensor(s) of a portable device generated by a user breathing on or near the device; and
processing the output(s) of both the touch screen and the motion sensor(s) to characterize a respiratory condition of the user.

6. A method as in any of claims 1 through 5 additionally comprising:
using a microphone on the device for detecting an audio signal simultaneous with other detected output(s);
further characterizing the respiratory condition using the audio signal.

7. A method comprising:
designing and using a battery or powered adjustable air pressure computer-like stylus to create an air pressure signal that will utilize claims 1 through 6 to develop a more sensitive and flexible Human-to-Device or Machine/Robot-to-Device interface.
